(19) United States
(12) Patent Application Publication
(10) Pub. No.: US 2022/0202307 A1
(43) Pub. Date: Jun. 30, 2022

(54) METHOD AND SYSTEM FOR MULTI-ELECTRODE MONITORING OF INTERNAL ELECTRICAL IMPEDANCE OF A BIOLOGICAL OBJECT

(71) Applicant: RS MEDICAL MONITORING LTD., Bnei brak (IL)

(72) Inventors: MICHAEL SHOCHAT, Matan (IL); Schmuel Gummer, Tel Aviv (IL)

(73) Assignee: RS MEDICAL MONITORING LTD., Bnei brak (IL)

(21) Appl. No.: 17/305,511

(22) Filed: Jul. 8, 2021

Related U.S. Application Data
(63) Continuation of application No. 15/561,052, filed on Sep. 24, 2017, now abandoned.

(51) Int. Cl. A61B 5/0535; A61B 5/00; A61B 5/0531; A61B 5/276

(52) U.S. Cl. CPC A61B 5/0535; A61B 5/7221; A61B 5/282; A61B 5/276; A61B 5/4878; A61B 5/0531

(57) ABSTRACT

A method and system for multi-electrode monitoring of an internal electrical impendance of a biological object using placing two arrays of electrodes on opposite sides of the biological object, wherein each of said two arrays comprise at least two spaced apart electrodes; performing session of measurements comprising imposing and alternating electrical current between pairs of said electrodes and obtaining voltage signals representative of a voltage drop thereon; calculating values of skin-electrode resistance for all said electrodes; comparing said calculated values of skin-electrode resistance therebetween, wherein result of the comparison exceeding a predetermined threshold value being representative of a potential failure in at least one of said electrodes.

### TECHNOLOGICAL FIELD AND BACKGROUND

The present invention relates to noninvasive biological techniques and, more particularly, to a multi-electrodes methods and devices for measuring and/or monitoring an internal electrical impedance of a portion of a biological object, such as the lung(s).

Fluid buildup in biological object is associated with many diseases, notably diseases of the heart. An important example of fluid buildup associated with heart disease is acute congestion or edema of the lungs. Because lung fluids usually have better electric conductivity than surrounding tissues, changes in liquids volume can be detected by the technique of impedance plethysmography. Changes in liquid volume can be detected by measurement electrical impedance of whole body or organ of interest.

Clinical signs of pulmonary edema (PED) are appeared on physical examination only after significant lung fluid accumulation and therefore are not sufficiently sensitive to allow clinical monitoring of Heart Failure patients. A decrease in lung impedance (LI) reflects an increase in lung fluid content and may herald evolving PED at very early stage and indicate the need to initiate pre-emptive therapy.

The monitoring of liquid changes within a biological object using two electrodes, one either side of the biological object, is well known in the art. However, this method has proved to be unfit for prolonged monitoring due to the drift of skin-to-electrode contact layer resistance. During prolonged contact between electrode and skin “skin-electrode” impedance is changed as a result of electrolyte penetration to zone of contact and as a result common impedance between two electrodes on both sides of the chest is also changed. Values of “skin-electrode” impedance variations usually much higher than variations of LI during evolving PED, and to this end measured common (total) impedance between two electrodes be indicative mainly of variation of “skin-electrode” impedance and not or only in small proportion in impedance of internal part of biological object or its portion as for example lung impedance or brain impedance.

A method for overcoming this problem was developed by Kubicek et al. (Annals of the New York Academy of Sciences, 1970, 170(2):724-32; U.S. Pat. No. 3,340,867, reissued as Re. 30,101). Related U.S. patents include Asrican (U.S. Pat. No. 3,874,368), Smith (U.S. Pat. No. 3,971,365), Matsuo (U.S. Pat. No. 4,116,231) and Itoh (U.S. Pat. No. 4,269,195). The method of Kubicek et al. uses a tetra-polar electrode system whereby the outer electrodes establish a current field through the chest. The inner voltage pickup electrodes are placed as accurately as is clinically possible at the base of the neck and at the level of the diaphragm. This method regards the entire portion of the chest between the electrodes as a solid cylinder with uniform parallel current fields passing through it. However, because this system measures the impedance of the entire chest, and because a large part of the electrical field is concentrated in the surface tissues and aorta, this method is not sufficiently specific for measuring variation of liquid levels in the lungs and has low sensitivity: 50 ml per Kg of body weight (Y. R. Berman, W. L. Schutz, Archives of Surgery, 1971. V. 102:61-64). It should be noted that such sensitivity has proved to be insufficient for obtaining a significant difference between impedance values in patients without pulmonary edema to those with an edema of average severity (A. Fein et al., Circulation, 1979, 60 (5):1156-60). In their report on the conference in 1979 concerning measuring the change in the amount of liquid in the lungs (Critical Care Medicine, 1980, 8(12):752-9), N. C. Staub and J. C. Hogg summarize the discussion on the reports concerning the reports on the method of Kubicek et al. for measuring thoracic bio-impedance. They conclude that the boundaries of the normal values are too wide, and the sensitivity of the method is lower than the possibilities of clinical observation and radiological analysis, even when the edema is considered to be severe. It is indicative that, in a paper six years later by N. C. Staub (Chest. 1986, 90 (4):588-94), this method is not mentioned at all.

Another method for measuring liquid volume in the lungs is the focusing electrode bridge method of Severinghaus (U.S. Pat. No. 3,750,649). This method uses two electrodes located either side of the thorax, on the left and right axillary regions. Severinghaus believed that part of the electrical field was concentrated in surface tissues around the thorax and therefore designed special electrodes to focus the field through the thorax. This method does not solve the problems associated with the drift in the skin-to-electrode resistance described above. An additional problem is the cumbersome nature of the large electrodes required. A review by M. Miniati et al. (Critical Care Medicine, 1987, 15 (12):1146-54) characterizes both the method of Kubicek et al. and the method of Severinghaus as “insufficiently sensitive, accurate, and reproducible to be used successfully in the clinical setting” (p. 1146).

Other notable recent work in measuring the impedance of a portion of the body includes the tomographic methods and apparatuses of Bai et al. (U.S. Pat. No. 4,486,835) and Zadehkoochak et al. (U.S. Pat. No. 5,465,730). In the form described, however, tomographic methods are based on relatively instantaneous measurements, and therefore are not affected by electrode drift. If tomographic methods were to be used for long-term monitoring of pulmonary edema, they would be as subject to problems of skin-to-electrode impedance drift as the other prior art methods.

FIG. 1 schematically illustrates components of Transthoracic Impedance (TTI) with depiction of the thoracic cross-section. Measurement electrodes 3 and 3′ are placed on opposite sides of the thorax of a patient. Transthoracic impedance (TTI) 2 generally composed by three components: Internal Thoracic Impedance (ITI) 1 that nearly equals inherent lung impedance (LI) plus high skin-electrode impedances at the front 3 and at the back of the chest 3′. Internal Thoracic Impedance of patients without congestion (normal state) is relatively low 25-120Ω (mean 60Ω) that could be decreased by 15-50% with the development of pulmonary congestion or edema. The skin-electrode impedance is relatively high (200-800Ω) and its value could change as a result of slow variations in skin ionic balance throughout monitoring of several hours' duration. It is also depended from individual characteristics of patients such as skin consistent, weight, height and sex. The absolute values of TTI are typically between 450-1700Ω. The magnitude of ITI decreasing during pulmonary congestion or edema development is approximately 15-50% from normal baseline level (25-120Ω). It means that ITI decreases by 4-60Ω during pulmonary congestion or edema development. Obviously, this change in ITI represents only a small part (1-4%) of the high TTI and is, therefore, barely measurable.

In order to improve sensitivity of ITI measurements in U.S. Pat. No. 5,749,369 Rabinovich et al was proposed a technique which enables subtracting the skin-electrode resistance of measurement electrodes including its drifts from whole thoracic impedance while measuring internal impedance of the biological object.

This technique uses measuring “skin-electrode” impedance for biological object on both sides of biological object and subtracting it from common (total) TTI. By such way ITI of biological object can be calculated more accurately during long monitoring period for each session of measurements (session means here and further below one measurement for each circuit) during monitoring. ITI impedance calculated according to this technique will not be affected by “skin-electrode” drift because “skin-electrode” impedance is calculated for each ITI measurements.

Subtracting calculated skin-electrode impedance value from transthoracic impedance TTI provides a solution for a problem of skin-electrode impedance drift and improves sensitivity of ITI measurement.

The technique disclosed in the U.S. Pat. No. 5,749,369 using multi-electrode system for impedance plethysmography with relative immunity to skin-electrode contact resistance drift. The technique uses multiple electrodes defining one measurement and six (plurality) reference electrical circuits. Electrical impedances of all circuits are measured and internal impedance of the biological object is calculated therefrom based on some physical assumptions as will be explained furtherbelow.

The Edema Guard Monitor (EGM) model RS-207 (RS Medical Monitoring, Israel) was developed according to the U.S. Pat. No. 5,749,369 to address the skin-electrode contact resistance drift monitoring problems. It is designed to noninvasively monitor with better signal-to-noise characteristics than other noninvasive devices.

This system solved the problems of high skin-to-electrode impedances and their drifts during prolonged monitoring by separating ITI from TTI by reducing (subtracting) skin-electrode impedance drift at the time of each ITI measurement.

However, this technique uses calculating value of the skin-electrode resistance drift of measurement electrodes only, i.e. electrodes forming “measurement circuit” not taking into consideration skin-electrode contacts of “reference” electrodes that constitute “reference circuits”.

In addition, this technique is based on assumption that absolute values of all skin-electrode contacts and their drift are relatively of the same degree. During of use RS-207 system the inventors have found that there are at least two situations when this physical assumption is not correct. It could be a case, when one or more electrodes (measurement or/and reference) is faulty, e.g. broken and own electrode impedance is very high. In addition, entire cycle of impedance measurements with multiple-electrode system could take few seconds and patients usually make uncontrolled movements of body or especially extremities. As result of such movements contact between electrode and skin could be changes which in turn induce dramatic changes in skin-electrode resistance. In such situation obtained impedances for measurement or/and reference circuits can varies dramatically and not in the same degree for different electrodes. In both cases accuracy of measurements could be significantly reduced.

There is accordingly a need in the art for a novel approach for noninvasive technique solving the problem of limited accuracy/sensitivity to detect small ITI changes occurring during the early stage of interstitial Edema when preventive treatment is desirable and most effective.

General Description

Thus, according to one broad aspect of the invention, there is provided a method for multi-electrode monitoring of an internal electrical impedance of a biological object, comprising the steps of placing two arrays of electrodes on opposite sides of the biological object, wherein each of said two arrays comprise at least two spaced apart electrodes performing session of measurements comprising imposing an alternating electrical current between pairs of said electrodes and obtaining voltage signals representative of a voltage drop thereon; calculating values of skin-electrode resistance for all said electrodes; and comparing said calculated values of skin-electrode resistance therebetween, wherein result of the comparison exceeding a predetermined threshold value being representative of a potential failure in at least one of said electrodes.

Preferably, the steps ii-iv are repeated in case when the result of the comparison exceed a predetermined threshold value. Correctness of the measurements of the measurement session or faultless of at least one of the electrodes based on result of the comparison could be defined.

Measurement session could be denied or accepted and faulty electrode(s) could be replaced.

More specifically, the present invention is useful for monitoring Internal Thoracic Impedance (ITI) and the pre-determined threshold value in tha case is about 150 Ohm.

In some embodiments, theealternating electrical current has a value from 0.5 to 5 mA, or more specifically from 1 to 2 mA.

More specifically, alternating electrical current has a frequency from 50 to 200 KHz and could be of any periodic waveform.

According to yet another broad aspect of the invention, there is provided system for monitoring an internal electrical impedance of a biological object, comprising a plurality of electrodes, current source and a voltage measurement unit connected to an analog multiplexer operable for alternately connecting said current source and said voltage measurement unit to form predetermined sets of said electrodes, a control unit with data processing utility for carrying out calculations of a skin-electrode resistance for all said plurality of electrodes and comparing of said calculated therebetween.

In some embodiments the current source provides alternating electrical current from 0.5 to 5 mA and more specifically from 1 to 2 mA.

Predetermined sets of the electrodes preferably comprises a pair of electrodes.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates components of Transthoracic Impedance (TTI) with depiction of the thoracic cross-section,

FIG. 2 is a schematic illustration of two arrays of electrodes of impedance plethysmography device used to monitor pulmonary edema;

FIG. 3 is a partial schematic illustration of device according to one preferred embodiment;

FIG. 4A illustrates biological object (human body) equivalent circuit model;

FIG. 4B illustrates equivalent circuit model of system including human body faulty electrode(s) of impedance plethysmography device;

FIG. 5 schematically illustrates electric circuitry composed by plethysmography device electrodes and biological object (skin/lung) according to one preferred embodiment of the present invention;

FIG. 6 schematically illustrates electric circuitry composed by plethysmography device electrodes and biological object (skin/lung) according to another preferred embodiment of the present invention;

FIG. 7 shows a flow diagram of an example of the invention according to one preferred embodiment; and

FIG. 8 is a schematic block diagram of the system according to the present invention.

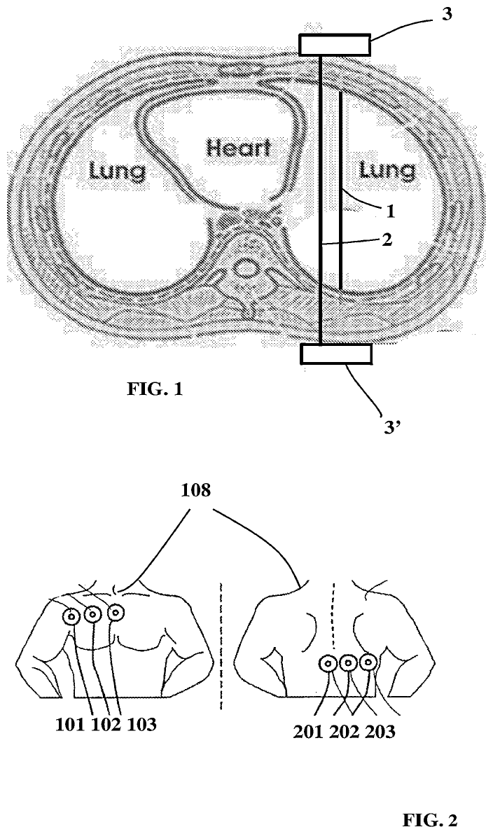

### DETAILED DESCRIPTION OF EMBODIMENTS

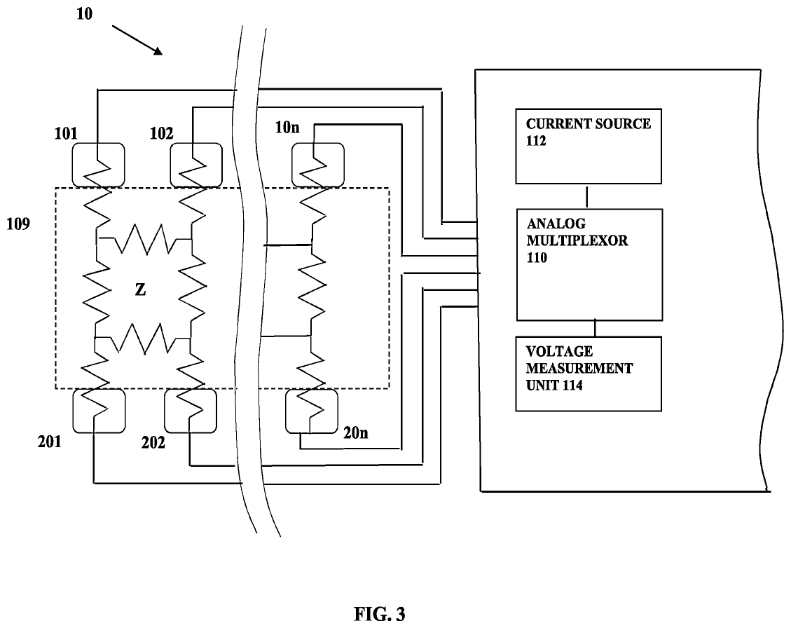

Reference is made to FIGS. 2 and 3 exemplifying general features of multi-electrode system for measurements of internal impedance of biological object.

FIG. 2 more specifically illustrates two arrays of electrodes 101-103 and 201-203 of impedance plethysmography device placed on opposite sides of thorax 108 for monitoring pulmonary edema. Each array as illustrated comprises three spaced apart electrodes, but also could comprise two or more than 3 electrodes.

Refereeing to FIG. 3 illustrating more general example of a multi-electrode system 10 for measuring internal electrical impedance of biological object 109 comprising two arrays of spaced apart electrodes 101-102 and 104-202 placed on opposite sides of biological object 109. As shown by dash lines in FIG. 2 each array of electrodes could include any desired number of electrodes n placed on opposite sides of biological object 109. An analog multiplexer 110 controlled by control unit CU performs selective connecting of electrodes to a current source 112 and a voltage measurement unit 114. Selective connecting of electrodes 101-202 (20n) could form pre-determined sets of electrodes comprising desired number of electrodes of any one or both arrays of electrodes.

Sets formed by electrodes of array placed on the same side of biological object 108 i.e. 101-102 (10n) or 201-202 (20n) forms “reference” circuits and sets formed by at least two electrodes of different arrays form measurement circuits. Analog multiplexer 110 is capable to connect any desired combinations of electrodes 101-202 forming pre-determined sets. It should be noted that pre-determined sets of electrodes could comprise at least two electrodes of one or both arrays. Preferably, pre-determined sets could comprise a pair of electrodes.

In order to provide measurements of internal impedance, arrays could be placed on opposite sides of biological object, e.g. on opposite sides of a patient thorax in case of impedance plethysmography (as specifically illustrated specifically in FIG. 2). Preferably, each array could comprise three spaced apart electrodes.

Current source 112 supplies alternative electrical current of substantially identical intensity, e.g. of about 0.5-5 mA between electrodes of any predetermined set (e.g. electrodes of each pair of electrodes).

For impedance plethysmography preferably, current from about 1 mA to about 2 mA at a frequency of between about 50 KHz and about 200 KHz is used. Current of about 1 mA most preferably could be used. The term “frequency”, as used herein, refers to the fundamental frequency of a periodic waveform, so that the scope of the present invention includes alternating current of any periodic waveform, for example square, saw, etc. waves, and not just sinusoidal alternating current.

A voltage drop V across the measurement and reference circuits is measured by voltage measurement unit 114 while imposing an alternative current between circuit's electrodes. So obtained voltage signals V being representative of a voltage drop on circuit's electrodes. Generally, voltage drop across the measurement circuits being indicative of (proportional) a total impedance of the biological object and voltage drop across the reference circuits being indicative of (proportional) skin-electrode impedance.

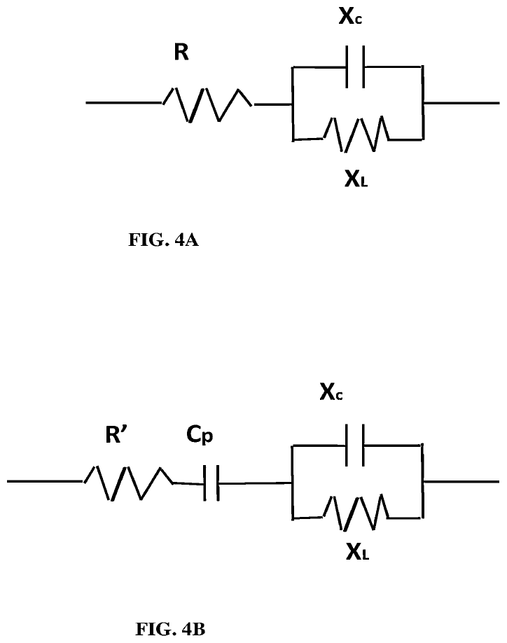

In order to better understand the subject matter that is disclosed herein and to exemplify how it may be carried out in practice, reference is now made to FIGS. 4A and B illustrating biological object (e.g. human body) equivalent circuit model and equivalent circuit model of system including human body and faulty electrode(s) of impedance plethysmography device accordingly.

As shown in FIG. 4A, impedance of biological tissue Z composed by active resistance component R and reactive components: capacitance—X_{c} and inductivity—Xₗ. Total impedance Z could be calculated as following:

Z=\sqrt{R²+(Xₗ−X_{c})²}

In case of plethysmography typical operation frequencies 50-500 KHz, capacitance—X_{c} and inductivity—Xₗ have small values, and their subtraction is practically equal to zero. Hence impedance Z of biological tissue is mostly defined by reactive resistance R (Z≈R).

As further illustrated in FIG. 4B, in the case of electrode failure active resistance component R′ could be significantly higher than for “faultless” electrode and also additional parasitic capacitance Cₚ could became noticeable. In such case, assumption that Z≈R in biological tissue might be incorrect and in turn obtained result might be useless.

Several factors can contribute to electrode failure: defective electrode which is fairly common occurrence, partial disconnection of electrode(s) due to patient movements, breath and/or perspiration, electrode degradation during monitoring period, etc.

The inventors found that in order to properly mitigate the faulty electrode(s) problem in multi-electrode plethysmography it is necessary to calculate values of skin-electrode resistance for all electrodes (i.e. composing reference and measurement circuits) and to compare calculated values therebetween upon completing all measurements of session (and before calculating of internal electrical impedance of a biological object). Result of the comparing will be representative of a potential failure in at least one of the electrodes and enables to identify the potentially failure electrode(s). A pre-determined threshold value of resistance difference could be further used to deny or accept a faultless of electrode(s) and correctness of measurements for measurement session. It was found by inventors that pre-determined threshold value preferably being about 150 Ohm. If skin-electrode resistance of at least one electrode appears to be higher at 150 Ohms comparing to other electrodes, the measurements should be repeated. If the same result is obtained for repeating measurement session(s) it means that electrode with high skin electrode resistance (>150 Ohms than others) is faulty and should be replaced. After replacement of faulty electrode(s) by new electrode(s) measurement session(s) including entire sequence should be performed and skin-electrode resistances should calculated and compared until obtaining acceptable result. Additional session(s) including calculation and comparison of values of skin-electrode resistance for all electrodes could be performed also for verification purposes.

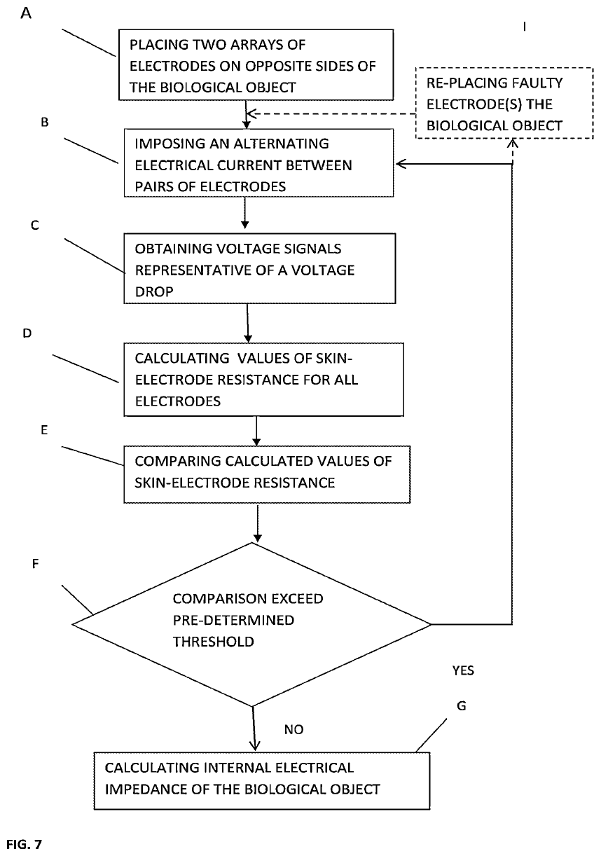

FIG. 7 shows a flow diagram of an example of the present invention. According to this example, two arrays of electrodes are placed on opposite sides of the biological object (step A), where preferably each of two arrays comprise at least two spaced apart electrodes. Then an alternating electrical current is imposed between pairs of the electrodes (step B) and voltage signals representative of a voltage drop on electrodes is obtained (step C). Further, values of skin-electrode resistance for all electrodes based on Ohm law are calculated (step D) and compared (step E).

The comparison could be performed between calculated values of skin-electrode resistance or relative to some reference value (e.g. calculated (average) or pre-set). If result of comparison do not exceed (step F) pre-determined threshold value (e.g. 150 Ohm in case of thorax plethysmography), the obtained signals are used in order to calculate internal electrical impedance of biological object (step G). Otherwise, measurements could be repeated (specifically steps B-C) and values of skin-electrode resistance for all electrodes based on Ohm law are calculated (step D) and compared (step E) once more. If result of comparing (stage F) for repeating measurement is differ from previous one and within acceptable value (e.g. <150 Ohms) it means that difference(s) between skin-electrode resistances on first measurement was a result of patient's movement and there is no electrode(s) faulty. To this end results of such measurements could be further used for calculation if Internal Lung Impedance. If results of comparison still do exceed pre-determined threshold value for one or more electrodes that means that the electrode(s) is faulty. In such case the faulty electrode(s) is re-placed (step I) and measurements are repeated (steps after step B). In some cases potentially faulty electrode could be replaced directly after comparison step F without repeating measurement steps B and C and calculating/comparison steps D-E.

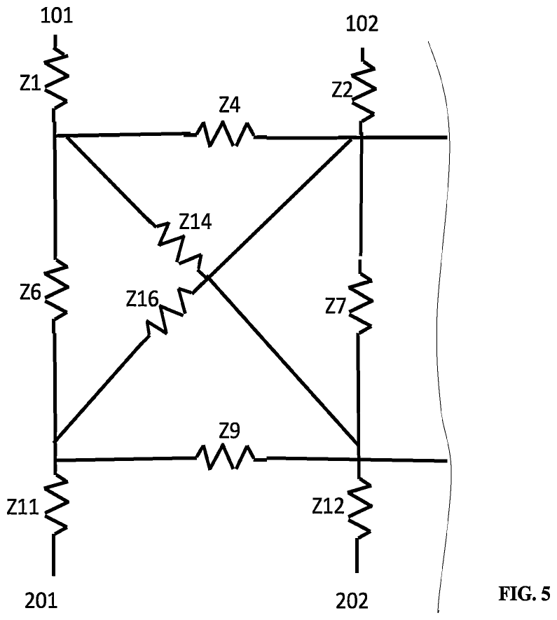

Referring to FIG. 5, the configuration and operation of the system 10 of the invention is now more specifically exemplified using effective electric circuit (circuitry) composed by plethysmography device electrodes and biological object (skin/lung). Effective electric circuit is based on physical assumption that the total impedance measured across two electrodes placed on opposite sides of the biological object is the sum of two impedances: the impedance of the skin-electrode contacts and the internal impedance of the body.

Generally the impedance Z_{M} of any measurement circuit formed by set of electrodes is the sum of the following impedances:

Z_{M}=Z_{IN}+Z_{A}+Z_{B} (A)

Where:

Z_{IN}—the internal impedance of biological object (e.g. ITI);

Z_{A}—“transition” impedance which includes the impedance of first electrode; the impedance of the skin-electrode contact of electrode; and skin impedance;

Z_{B}—“transition” impedance which includes the impedance of second electrode; the impedance of the skin-electrode contact of electrode; and skin impedance.

On the other hand, the impedance of any reference circuit formed by set of electrodes is representative of “transition” impedances only, i.e. the sum of the following impedances:

Z_{R}=Z_{A}+Z_{B} (B),

Where:

Z_{A}—“transition” impedance which includes the impedance of first electrode; the impedance of the skin-electrode contact of electrode; and skin impedance;

Z_{B}—“transition” impedance which includes the impedance of second electrode; the impedance of the skin-electrode contact of electrode; and skin impedance

Thus, internal impedance of biological object (e.g. ITI in our case) Z_{IN} could be calculated using voltage drops across measurement and reference circuits based on effective electric circuitry illustrated in FIG. 5. At least two measurement circuits formed by pairs (sets) comprising electrodes with substantially equal (similar) distance therebetween are used according to the present invention.

Measurement and reference circuits of the present invention could be characterized by the following impedances:

Z₁—impedance of electrode and skin-electrode contact of electrode 101;

Z₂—impedance of electrode and skin-electrode contact of electrode 102;

Z₁₁—impedance of electrode and skin-electrode contact of electrode 201;

Z₁₂—impedance of electrode and skin-electrode contact of electrode 202;

Z₄—skin impedance between electrode 101 and 102;

Z₉—skin impedance between electrode 201 and 201.

Sets of electrodes forming measurement circuits could comprise from minimum a pair up to all electrodes of both groups of electrodes while sets of electrodes forming reference circuits also could comprise from minimum a pair and up to all electrodes of one of the groups of electrodes.

According to the present invention, after placing two arrays of electrodes 101-102 and 201-202 on opposite sides of the biological object (e.g. patient's thorax) an alternating electrical current between pairs of electrodes is imposed and voltage signals representative of a voltage drop thereon are obtained. Furthermore, values of skin-electrode resistances (impedances) Z1, Z2, Z11 and Z12 for all electrodes 101-202 are calculated. Calculated values of resistances Z1, Z2, Z11 and Z12 are further compared therebetween. Result of the comparison will be representative of a potential failure in at least one of electrodes 101-202 and enables to identify the potential failure thereof. This result could be further used to deny or accept correctness of measurement (due to patient's movement) or faultless of electrode(s). Denying or acceptance could be based on exciding or non-exciding pre-determined threshold value of 150 Ohm in case of Internal Thoracic Impedance (ITI) monitoring.

In order to be able calculate internal impedance(s) of biological object (e.g. ITI) based on system of linear equations; appropriate number of measurements for each measurement session should be performed. Increasing the number of electrodes covering different areas of lung (with different current ways) and “averaging” obtained ITI measured results could reduce such negative effect caused by local non-uniformities or anomalies. In addition, multiple measurements used for calculating internal impedance(s) also could improve accuracy of obtained result. Preferably, six electrodes (three in each array) could be used. In that case maximal number of circuits formed by pair of electrodes of any of array is defined by number of combinations by pairs of all electrodes. For six electrode's scheme, e.g. used in Edema Guard Monitor (EGM) model RS-001 (RS Medical Monitoring, Israel) total number of sets (pairs) is 15 and accordingly 15 measurements providing 15 values of impedance M₁-M₁₅ for one measurement session could be performed. Impendences of each measurement or reference circuit could be calculated according to Ohm's Law based on the measured values of voltage drops.

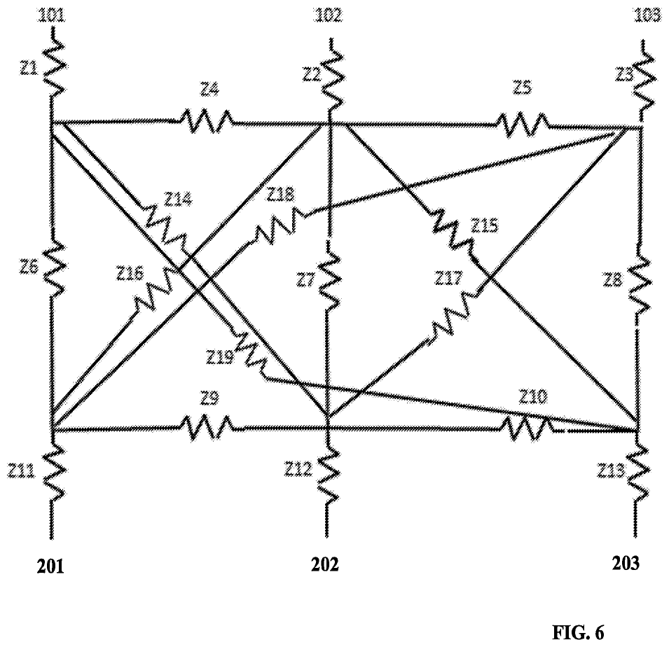

Various operations could be further performed with obtained values of impedances M₁-M_{15.} Referring to FIG. 6, for example, as only values of internal impedances Z₁₈ and Z₁₉ corresponding to measurement circuits defined by “uttermost” opposite electrodes could be calculated and used for characterizing internal impedance of the biological object (e.g. III). Since the inter-electrodes space in that case covers maximum biological object (e.g. lung) tissue these measurements could be most representative of variations of liquid amount within the lung tissue.

Performing maximal possible number of measurements for multi-electrode (six—in the present example) system could provide most efficient way of system operation as disclosed in PCT Patent Application No. PCT/2015/050048 in the name of the Applicant herein incorporated by reference in its entirety.

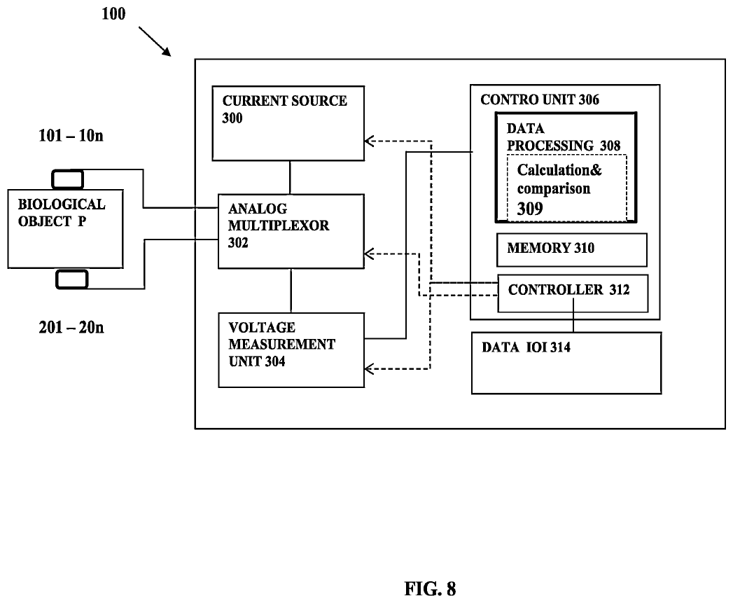

Reference is made to FIG. 8 exemplifying the configuration of system 10 of the present invention specifically useful for impedance plethysmography. As shown in FIG. 8 system 10 according to the present invention preferably includes: current source 300; analog multiplexer 302 for alternately connecting current source 300 to predetermined set of electrodes forming whether measurement or reference electrical circuits; a voltage measurement unit 304; a control unit 306 that includes data processing utility 308 for carrying out calculations; a data-storage unit (memory) 310 for storing data during measurement session(s) and entire monitoring period; a controller utility 312 for controlling the operation of units of system 100 such as current source 300, analog multiplexer 302, voltage measurement unit 304, etc.; data Input/Output interface—IOI 314. Data IOI 314 could include appropriate buttons, display, touch-screen enabling input of commands, data, etc. for operating the system 100 and displaying operating status of the system and measurement data. An alarm unit also could be provided (not-shown)

Data processing utility 308 could comprise appropriate SW and HW (sub-utilities) and is connectable to data-storage unit 310, data IOI 314 and optionally to alarm unit. These SW and HW provide operation of system 100 according to the method described above.

System 100 could be powered from external (e.g. AC) and/or internal (e.g. battery) sources by means of a power supply (not shown).

Voltage measurement unit 304 typically includes rectifier (not shown) for obtaining the absolute value of the signals representing the voltage drops and analog to digital A/D converter for converting analog signals to a digital form signal compatible with data processing utility 308.

When using a device according to the present invention, electrical source 300 is alternately connected to each of the electrical circuits formed by pre-determined sets, e.g. pairs of electrodes 101-106 shown in FIG. 8 by means of analog multiplexer (commutator) 302. Signal representing the voltage drop of a specific electrical circuit is fed voltage measurement unit 304 which preferably provides signal in digital form. The obtained digital signal is fed into control unit 306 for storing in data-storage unit (memory) 310 for further processing by data processing utility 308.

Control unit 306 orders analog multiplexer (commutator) 302 to form pre-determined number and configurations of measurement and reference circuits, e.g. 15 for six-electrodes scheme with two-electrodes sets of electrodes.

After data-storage unit (memory) 310 has received data from each of electrical circuits, appropriate impedance calculation&comparison sub-utility 309 of data processing utility 308 can calculate values of skin-electrode resistances (impedances) Z₁, Z₂, . . . Z₁₁ and Z₁₂, etc. for all electrodes 101-20n. Calculated values of impedances Z₁, . . . etc. are further compared therebetween. Result of the comparison will be representative of a potential failure in at least one of electrodes 101-203 and enables to identify the potential failure thereof. A pre-determined threshold value of resistance difference e.g. 150 Ohm could be set up and further used to deny or accept electrode(s). Information on faulty electrode(s) could be displayed or otherwise presented by data IOI 314 or optional additional display or indicators.

Upon completing the electrodes “check procedure” further the internal impedances Z_{IN} (e.g. values of Z₁₈ and Z₁₉) could be obtained according to the method as described above. Data processing utility 308 also could perform additional processing of multiple measurement results, e.g. comparison of values of Z₁₈ and Z₁₉ and their combining due to pre-set algorithm (averaging, weighing, etc.).

Preferably, when performing a monitoring of a biological object the process described above is carried out periodically, so that Data processing utility 308 can calculate the values of the internal impedance Z_{IN} as well as changes therein. The change in Z_{IN} may be calculated, for example, as the difference between the last value and the initial or previously measured value(s) or as a percentage therefrom. The results of the calculations could be transmitted to data IOI interface 14 and displayed by internal or external display, to data-storage unit (memory) 310, and to optional alarm unit.

In the event that the value of Z_{IN} has decreased below a critical value, and/or in the event that the change in Z_{IN} has exceeded a critical value, the alarm could be activated.

Data-storage unit (memory) 310 may provide data for analysis during the monitoring period so as to monitor the progress of the disease.

Thus, the present invention provides an effective and reliable technique for measuring the internal electrical impedance of a biological object and specifically Transthoracic impedance which can be used for effective monitoring in time of lung liquid volume status.

Those skilled in the art will readily appreciate that various modifications and changes can be applied to the embodiments of the invention hereinbefore described without departing from its scope defined in and by the appended claims.

We claim:

1. A method for multi-electrode monitoring of an internal electrical impedance of a biological object, comprising the steps of: placing two arrays of electrodes on opposite sides of the biological object, wherein each of said two arrays comprise at least two spaced apart electrodes; performing session of measurements comprising imposing an alternating electrical current between pairs of said electrodes and obtaining voltage signals representative of a voltage drop thereon; and calculating transition impedances related to the electrodes and the biological object using linear equations and based on predefined assumptions regarding one or more relationships between impedances that form the transition impedances.

2. A system for monitoring an internal electrical impedance of a biological object, comprising a plurality of electrodes, current source and a voltage measurement unit connected to an analog multiplexer operable for alternately connecting said current source and said voltage measurement unit to form predetermined sets of said electrodes, a control unit with data processing utility for calculating transition impedances related to the electrodes and the biological object using linear equations and based on predefined assumptions regarding one or more relationships between impedances that form the transition impedances.
